(19) United States
(12) Patent Application Publication
(10) Pub. No.: US 2014/0099257 A1
(43) Pub. Date: Apr. 10, 2014

(54) Androgen Suppression, Prostate-Specific Membrane Antigen and the Concept of Conditionally Enhanced Vulnerability

(71) Applicant: CORNELL UNIVERSITY, Ithaca, NY (US)

(72) Inventor: Neil H. BANDER, Chappaqua, NY (US)

(73) Assignee: CORNELL UNIVERSITY, Ithaca, NY (US)

(21) Appl. No.: 13/803,166

(22) Filed: Mar. 14, 2013

Related U.S. Application Data
(60) Provisional application No. 61/744,928, filed on Oct. 5, 2012.

(51) Int. Cl. C07K 16/40; C12Q 1/68; G01N 33/574

(52) U.S. Cl. CPC C07K 16/40; G01N 33/57492; C12Q 1/6886
USPC 424/1.49; 530/389.7; 530/391.7; 424/178.1; 424/158.1; 435/7.23; 435/6.13

(57) ABSTRACT

Anti-androgen therapies represent the cornerstone of prostate cancer (PC) treatment. Yet all PC patients ultimately fail efforts to rein in the androgen receptor (AR). This invention is based on the discovery that prostate-specific membrane antigen (PSMA), a highly PC-specific and clinically validated cell surface target, is AR-suppressed and up-regulated in PC as a result of hormonal manipulation. This up-regulation occurs in an unexpected timeframe and it occurs even in the castrate-resistant setting. As a result, hormonal therapy creates a state of conditionally enhanced vulnerability of PC to PSMA-targeted anti-cancer/cytotoxic agents that can be exploited by leveraging anti-AR therapy by the addition of PSMA-targeted agents. We demonstrate this conditionally enhanced vulnerability in a castrate-resistant animal model. The state of conditionally enhanced vulnerability may be relevant for other cancer targets and efforts to screen for them may improve other cancer therapies.

### BACKGROUND

This application claims priority to U.S. Provisional Patent Application No. 61/744,928, filed Oct. 5, 2012, the entirety of which is hereby incorporated by reference.

The androgen receptor (AR) is the key regulator of prostate glandular development and differentiation and androgen suppression is the backbone of advanced prostate cancer (PC) treatment. Recently, a new generation of more potent androgen suppressing agents have demonstrated meaningful clinical benefit (Danila et al. (2010); Scher et al. (2010)). But de novo or acquired resistance to these therapies suggests the continuing need to develop additional, complementary therapeutic approaches.

Prostate-specific membrane antigen (PSMA)/folate hydrolase 1 (FOLH1) is a plasma membrane receptor with many properties that make it a potentially valuable target: (1) its expression is highly specific for prostatic epithelium; (2) it is up-regulated in PC (Israeli et al. (1994); Wright et al. (1995); Troyer et al. (1995); and Sokoloff et al. (2000)); (3) it is expressed by virtually all PCs (Wright et al. (1995); Sweat et al. (1998); Bostwick et al. (1998); Mannweiler et al. (2009); Kusumi et al. (2008); and Ananias et al. (2009); (4) expression increases directly with tumor grade, stage and hormonal independence (Wright et al. (1995)); and (5) and PSMA functions as an internalizing cell surface receptor (Liu et al. (1997)).

While data suggests that androgen suppression up-regulates PSMA expression, there is some inconsistency in the published literature. On the one hand, Israeli et al (Israeli et al. (1994)) reported that androgen down-regulates PSMA in the LNCaP cell line and Wright et al. (Wright et al. (1996)) found that about half of primary PC specimens expressed higher levels of PSMA after hormonal therapy. On the other hand, Chang et al reported no increase in PSMA expression when comparing prostatectomy specimens from patients after 3 months of neo-adjuvant androgen suppression relative to those who did not receive hormonal therapy (Chang et al. (2000)) and Kusumi, et al (Kusumi et al. (2008)) reported that PSMA expression was decreased by hormonal therapy. More recently, Evans, et al (Evans et al. (2011)) reported PSMA expression was inversely regulated by androgens both in vitro and in animal xenograft models.

Thus, androgen suppression will almost certainly remain a critical component of any PC therapy and there have been previous suggestions of a relationship between androgen activity and PSMA expression. Nevertheless, the relationship between androgen and PSMA expression remains largely unknown and the potential of combining anti-androgen and anti-PSMA therapy also remains largely unknown.

### BRIEF SUMMARY OF THE INVENTION

The present invention is based on studies, which found, in a longitudinal and controlled manner across a panel of six human PC cell lines, that androgen suppression consistently led to PSMA up-regulation in all the lines and confirmed the recently published findings of Evans, et al (Evan et al. (2011)). The previously noted studies in the literature that failed to demonstrate anti-androgen up-regulation of PSMA (Kusumi et al. (2008); Wright et al. (1996); and Chang et al. (2000)) compared PSMA expression between independent groups of patients and likely were confounded by inter-patient variability of PSMA expression.

When PC cell lines were subjected to androgen suppression, it led to as much as an 80-fold increase in PSMA expression relative to its level in physiological concentrations of DHT. Nevertheless, while the directional changes in PSMA expression associated with changes in androgen axis activity were qualitatively identical among all the cell lines, the individual cell lines expressed widely different quantitative levels of PSMA even under identical concentrations of DHT suggesting that PSMA expression is not solely a function of androgen concentration. The variability of PSMA level notwithstanding, PSMA represents a useful cellular biomarker to monitor or measure AR functional activity, however, a static reading of PSMA level will be less informative than intra-patient comparisons of serial (e.g., pre- and post-intervention) readings. In addition, studies show that the change in PSMA expression between androgen-intact and androgen-suppressed states correlated with the level of AR expression of the cell line—i.e., a lower absolute AR level was associated with a narrower dynamic range of PSMA expression as a result of androgen fluxes, and vice versa. Such an evaluation may provide a means to measure AR expression level in vivo.

With regard to the temporal response of androgen-regulated genes, Nelson, et al (Nelson et al. (2002)) reported 4 temporal patterns within a timeframe up to 48 hours. Interestingly, our studies found that after androgen withdrawal, the increase in PSMA expression is delayed, taking approximately 2 weeks to reach a peak. This finding explains why studies of androgen-regulated gene expression profiles that utilized androgen exposure/withdrawal intervals of ≦48 hours (Nelson et al. (2002); Wang et al. (2009); and Hendriksen et al. (2006)) have missed androgen regulation of PSMA/FOLH1 whereas a study that assessed such profile changes over a longer interval of androgen withdrawal (Mostaghel et al. (2007)) identified PSMA/FOLH1 as one of the most highly up-regulated genes/proteins and the single highest up-regulated plasma membrane protein. Based on our temporal findings, use of intervals shorter than 1-2 weeks may miss up-regulation of PSMA/FOLH1 and potentially other AR-repressed genes. The delay in PSMA de-repression suggests that AR binds tightly to PSMA regulatory elements and has a slow off-rate or half-life. The nature of the anti-androgen intervention and its respective ability to displace AR from these regulatory elements may affect the kinetics of PSMA expression.

PSA and PSMA both represent biomarkers of androgen activity, although the former is induced while the latter is repressed by androgens. In addition, while PSA is sampled in plasma or serum and represents the average output of all lesions, PSMA expression can be used as a pharamcodynamic biomarker of androgen activity at the level of the individual cell or lesion. For example, ex vivo analysis of captured circulating tumor cells (CTCs; Miyamato et al. (2012)) or in vivo patient imaging with PSMA-targeted agents can identify PSMA changes indicative of changes in androgen axis activity (Evans et al. (2011)). Along with collaborators, we recently initiated a clinical trial (NCT01543659) with ⁸⁹Zirconium-J591, a PSMA-targeted PET agent capable of quantitative reporting of PSMA levels in vivo (Holland et al. (2010); and Evans et al. (2011)).

Lastly, this invention demonstrates that the relationship between androgen suppression and PSMA expression can be exploited to create a state of “conditionally enhanced vulnerability.” That is, the condition of androgen suppression drives increased target (PSMA) expression that, in turn, results in enhanced tumor cell vulnerability to a (PSMA)-targeted therapeutic agent. The CWR22Rv1 case was chosen to study this as it represents a particularly high hurdle: it is castrate-resistant, one of the lowest PSMA-expressing PC cell lines, expresses PSMA heterogeneously, expresses low levels of AR, and is among the lowest PSMA up-regulating cell lines when androgen-suppressed. Interestingly, the castration-induced doubling in anti-tumor efficacy of a PSMA-targeted agent demonstrated in the CWR22Rv1 model closely approximates the post-castration increase in amount of J591 targeted antibody measured in vivo by PET imaging (Evans et al. (2011)). In tumors that demonstrate a higher multiple of PSMA up-regulation, one would anticipate an even greater enhancement in PSMA-targeted therapeutic efficacy. And, in castrate-sensitive tumors, one would anticipate still greater efficacy resulting from the independent effects of the respective agents as well as the benefit derived from their interaction.

This invention represents the first example of pharmacologic modulation of a target to enhance a coordinate targeted therapeutic. In an era of targeted antibody- or ligand-drug conjugates, screens may be readily set up that potentially identify agents that lead to target up-regulation and conditionally enhanced vulnerability. While other examples of conditional vulnerability may be found, the case of AR-PSMA is particularly fortuitous given the central role of androgen suppression in PC treatment, the specificity of PSMA, and the resulting increase in PSMA receptor expression—all of which combine to create a unique therapeutic opportunity that can be achieved by co-targeting these two receptors. In this case, the efficacy directly increased, as is the therapeutic index, by increasing target expression by the androgen-regulated cancer cell but not by AR-negative non-target/normal cells.

The biological features of PSMA provide a significant opportunity to leverage and build upon anti-androgen therapies in PC. We have begun to clinically translate this co-targeting opportunity by combining anti-androgen approaches plus PSMA-targeted cytotoxics, factoring in their temporal relationship, into our PSMA-targeted antibody therapy trials (e.g., NCT00859781). While there are efforts underway to elucidate mechanisms of resistance to anti-androgen approaches, one should not overlook an opportunity provided by anti-androgen-induced enhanced tumor sensitivity.

Thus, the invention is based, in part, on the foregoing discovery—that an inverse relationship exists between androgen level and PSMA expression. Any patient having adequate expression levels of PSMA can be targeted for treatment by an anti-PSMA-targeted drug. Accordingly, because of the inverse relationship between androgen levels and PSMA expression, patients having low level of androgen are likely to also have elevated expression of PSMA, which makes them ideal candidates for PSMA-targeted antibody therapy.

This invention is also based, in part, on the discovery that, because of the inverse relationship between androgen levels and PSMA expression, a combination of anti-androgen therapy and anti-PSMA antibody therapy is synergistically efficacious in treating prostate cancer; the anti-androgen treatment up-regulates expression of the PSMA target thereby leading to delivery of an increased quantity of the anti-PSMA-targeted drug. There are a number of anti-androgen therapies, including, but not limited to, hormonal therapy (i.e., medical or chemical castration) or surgical castration therapy. One of the unexpected findings is that even in patients who are so-called “castrate-resistant”—for whom castration therapy would not be expected to induce a therapeutic response nor to have any effect on PSMA expression—castration nonetheless does up-regulate PSMA expression, and therefore results in an even better anti-PSMA therapeutic response. That is, anti-PSMA targeted therapy is not only useful in treating castrate-sensitive (i.e., androgen-sensitive/androgen-responsive) patients, but it is also useful in treating castrate-resistant patients, i.e., patients for whom anti-androgen therapy ordinarily would not be expected to be beneficial. Thus, a surprising finding of this invention is that castrate-resistant prostate cancer patients, who by definition are not responsive to anti-androgen therapy, nevertheless benefit from anti-androgen therapy when combined with anti-PSMA antibody treatment.

The claimed invention is also directed to a method of identifying a test agent that increases the level of PSMA expression on a prostate cancer. Such test agents might also be agents that reduce androgen levels.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a comparison of PSMA expression in presence and absence of DHT. Quantitative imunoblots using Li-cor technology. FIG. 1A shows gels representing cells grown in 10 nM DHT (upper panel) or absence of DHT (after charcoal-stripping of FCS; lower panel). Optical density of the PSMA band was indexed to the β-actin band in each lane. FIG. 1B shows the ratio of PSMA/β-actin, in presence or absence of DHT, is plotted for each cell line. The relative increase in PSMA expression resulting from androgen withdrawal is indicated. The cell lines are shown at the bottom of Panel 1B. In this figure, MDA=MDA−Pca2b; and CWR=CWR22Rv1.

FIG. 2 shows that androgen withdrawal up-regulates PSMA expression. FIG. 2A shows that a FACS analysis demonstrates that LNCaP, with mutated AR, has elevated PSMA level at baseline in standard FCS-supplemented medium. Use of charcoal-stripped FCS further up-regulates PSMA 7-9-fold, peaking at 2 weeks. The lower cell number at 3 weeks reflects cell loss due to steroid starvation. Mean florescence intensity (MFI) readings are shown above each histogram. FIG. 2B shows a dose response of PSMA expression by LNCaP cells grown for 2 weeks with varying levels of androgens. Decreasing steroid concentration in this experiment led to a maximal increase in PSMA of 5.4-fold.

FIG. 3 shows that PSMA expression is inversely related to AR level. Panel 3A shows that transfection of AR into LNCaP (LNCaP-AR) results in down-regulation of PSMA expression by approximately 80% as measured by FACS. Conversely, AR-siRNA treatment silences AR and up-regulates PSMA expression in LNCaP and CWR22Rv1 at 48 hours (FIG. 3B) and in MDA-Pca-2b and LAPC-4 cells at 4 days (FIG. 3C).

FIG. 4 shows immunohistochemical (IHC) assessment of PSMA expression before and after castration. FIG. 4A shows baseline PSMA expression of CWR22Rv1 xenograft prior to castration. FIGS. 4B-4D show PSMA expression at 1 week (FIG. 4B), 2 weeks (FIG. 4C), and 4 weeks (FIG. 4D) post-castration.

FIG. 5 shows the effect of combining castration plus a PSMA-targeted cytotoxin. After establishment of growing tumors, mice received a 3 dose regimen at 2 week intervals (days 0, 14, 28). Controls included PBS-treated intact mice and PBS-treated castrate mice; both groups had overlapping growth curves consistent with the androgen-independent nature of CWR22Rv1. A third control group of mice was treated with unconjugated anti-PSMA mAb J591 plus free duocarmycin at equivalent doses to the highest dose (5 mg/kg) ADC group. The PBS-treated groups of mice demonstrated rapid tumor growth such that they required sacrifice by the end of the dosing period (day 28). The group treated with unconjugated mAb J591 and free duocarmycin showed minimal slowing of tumor growth relative to the PBS-treated control groups. Groups of animals treated with the J591/PSMA-targeted ADC at doses of 1, 3 or 5 mg/kg demonstrated a clear dose-response effect. While castration had no growth inhibitory effect on this castrate-resistant tumor model, the group treated with castration plus 3 mg/kg had an anti-tumor effect equivalent to approximately a 2-fold higher dose of ADC in the non-castrate animals. FIG. 5A shows the mean tumor volume and FIG. 5B shows photographs of the mice.

FIG. 6 shows that silencing AR up-regulates PSMA. FACS analysis of LNCaP (FIG. 6A), MDA-Pca-2b (FIG. 6B), and LAPC-4 cells (FIG. 6C) treated with AR-siRNA (blue line), non-targeted-siRNA (red line) and untreated control (green line). Gray histogram is secondary antibody-only negative control. In all cases, AR-siRNA silenced AR and up-regulated PSMA; the non-targeted-siRNA control did not affect expression of either AR or PSMA.

FIG. 7 shows the effect of combining castration plus a PSMA-targeted cytotoxin. Similar to experiment shown in FIG. 5, after establishment of growing tumors, mice received a 3 dose regimen at 2 week intervals (days 0, 14, and 28). Controls included PBS-treated intact mice, PBS-treated castrate mice, and a third control group of mice treated with unconjugated anti-PSMA mAb J591 plus free duocarmycin at equivalent doses to the highest dose (5 mg/kg) ADC group. The PBS-treated groups of mice demonstrated rapid tumor growth such that they required sacrifice by the end of the dosing period (day 28). The group treated with unconjugated mAb J591 and free duocarmycin showed minimal slowing of tumor growth relative to the PBS-treated control groups. Groups of animals treated with the J591/PSMA-targeted ADC at doses of 1, 3, or 5 mg/kg demonstrated a clear dose-response effect. While castration had no growth inhibitory effect on this castrate-resistant tumor model, the group treated with castration plus 3 mg/kg had an anti-tumor effect substantially greater than a higher dose of ADC in the non-castrate animals.

### EXAMPLES

As discussed previously, androgen ablation is the cornerstone of advanced prostate cancer (PC) treatment. Prostate-specific membrane antigen (PSMA), another target of interest in PC, has variously been reported to be regulated by androgens. These examples clarify this relationship and explore the potential utility of combined targeting of AR and PSMA. In general, expression of PSMA by seven established PC cell lines and in a xenograft model was studied by FACS, western blot, and immunohistochemistry (IHC) in androgen-intact, androgen-deprived, and AR-silenced conditions. The effect of combining castration with PSMA-targeted antibody-drug conjugates (ADC) were studied in a castrate-resistant xenograft model.

### Androgen Axis Activity Inversely Regulates PSMA Expression

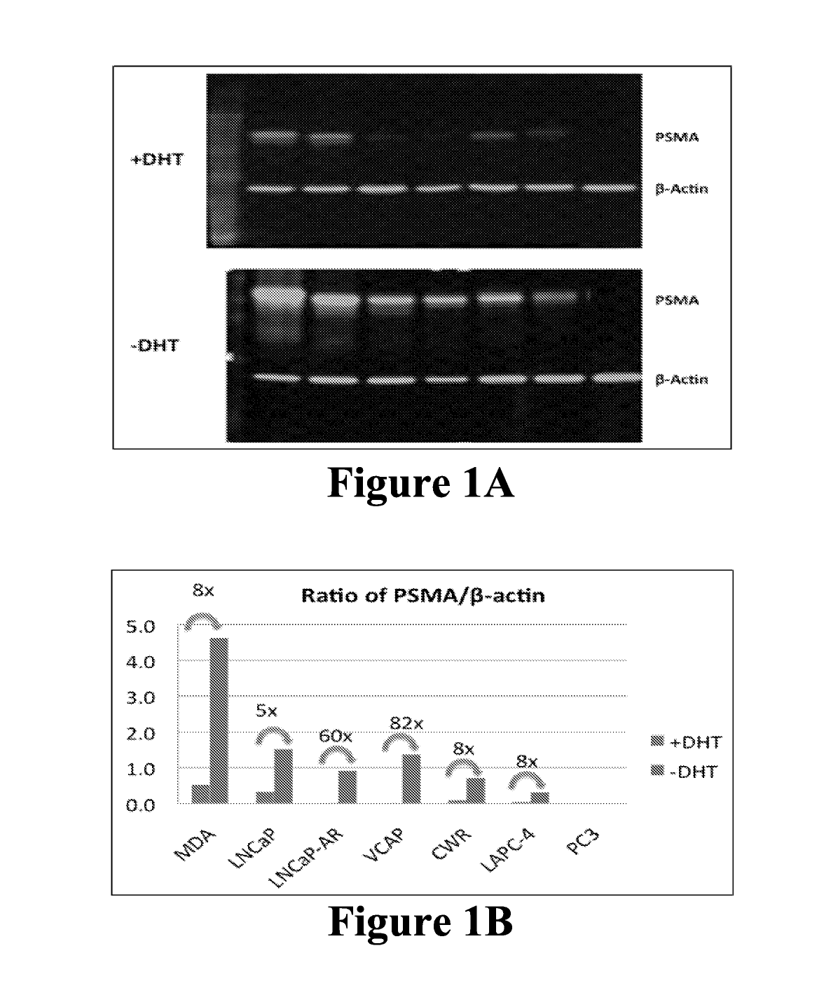

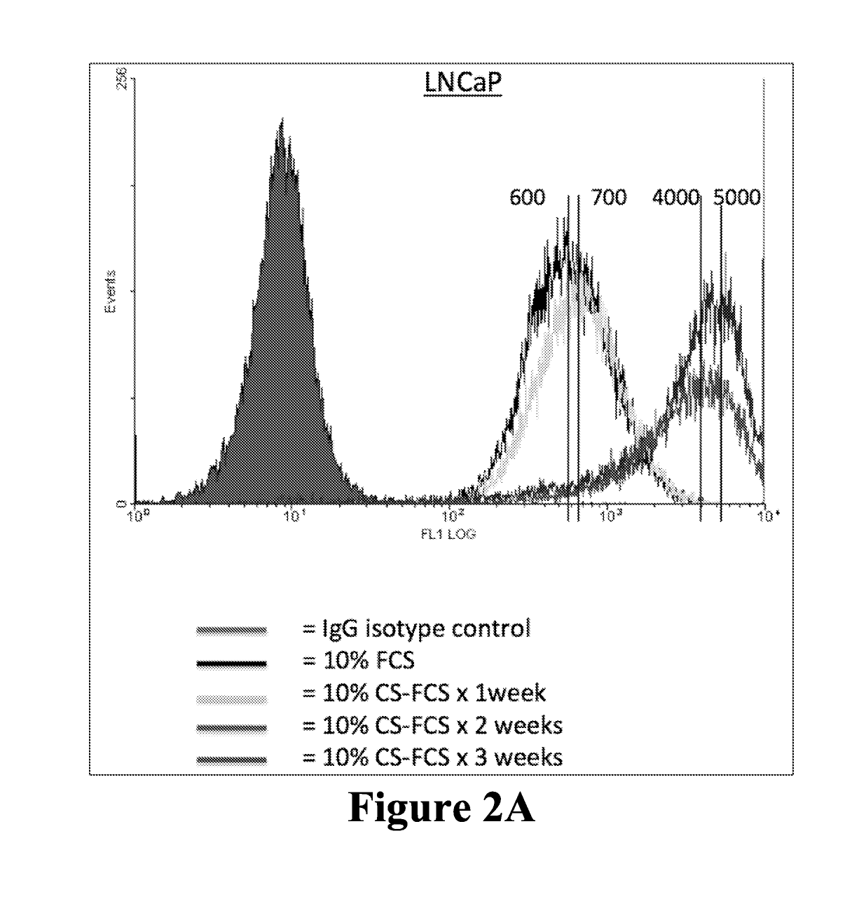

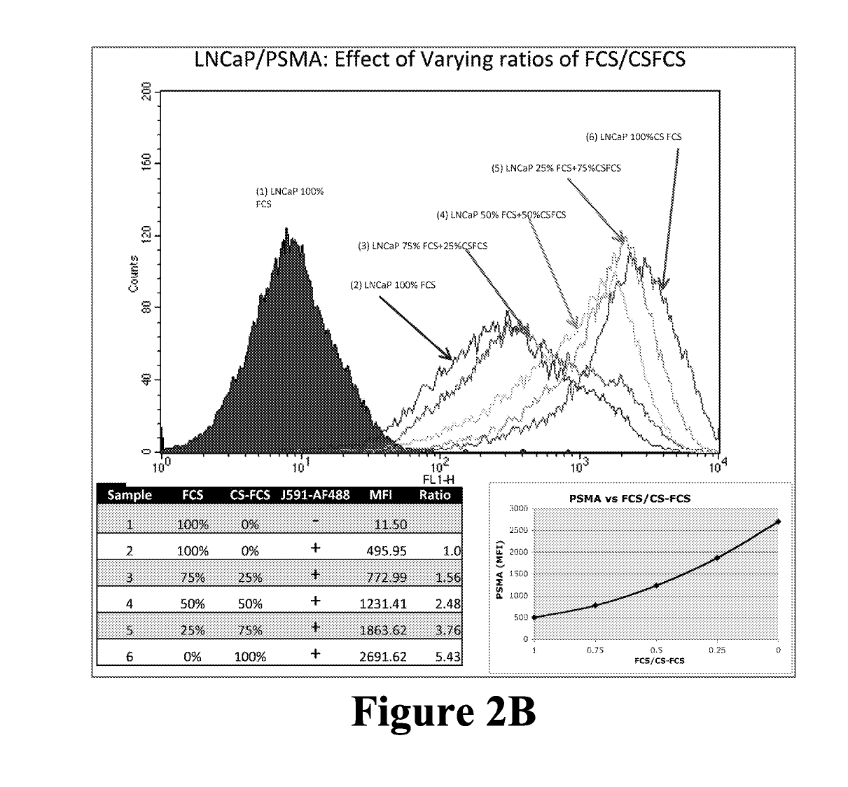

Charcoal-stripping the growth medium of 6 PC cell lines led to PSMA up-regulation between 4.6-81.6-fold relative to that in physiological levels of DHT (FIG. 1). Evaluation of the time course of the up-regulation revealed a delay in onset of several days to 1 week with peak expression found at 2 weeks (FIG. 2). The dose-response of PSMA expression relative to media steroid concentration is shown in FIG. 2(b). As measured by FACS mean fluorescence intensity (MFI), PSMA expression increases approximately linearly relative to decreasing concentration of steroids in the growth medium.

For western blots, cells were lysed with Cell Lysis Buffer (Cell Signaling Technology, Danvers, Mass.) containing 1 mM phenylmethylsulphonyl fluoride (EMD Chemicals, Gibbstown, N.J.). Equal amounts of protein were applied in each well on a 10% Tris-HCl gel (Bio-Rad Laboratories, Hercules, Calif.). The proteins were transferred onto Immobilon-P Membranes (Millipore, Billerica, Mass.), after which the filters were probed with the following reagents: murine anti-PSMA mAb J591, murine mAb anti-AR (AR441), rabbit anti-human AR, murine mAb anti-human beta-actin, and/or goat polyclonal anti-GAPDH. For quantitative western blots, the Li-cor Odyssey Infrared Imaging System (Lincoln, Nebr.) was used. With this system, two different proteins of the same molecular weight (e.g., PSMA and AR) can be detected simultaneously and quantified on the same blot using two different antibodies from two different species (mouse and rabbit) followed by detection with two IRDye labeled secondary antibodies. Anti-beta-actin is used as a loading reference Millipore Immobilon-FL PVDF membranes were used following Licor's recommendations. muJ591 anti-PSMA 1 ug/ml, rabbit anti-human AR 1:500 and mouse anti-human beta-actin 1:10,000 in 5% dry milk/PBST were combined and incubated simultaneously with the membranes for 1 hr. After washing, IRDye 800CW-goat anti-mouse secondary antibody (1:10,000) and IRDye 680LT-goat anti-rabbit secondary antibody (1:20,000) in 5% dry milk/PBST were combined and incubated simultaneously with the membranes. After washing, the membranes were scanned and the bands were quantified with the Odyssey Infrared Imaging System.

Numerous cell lines were used in these examples. Human prostate cancer cell lines, LNCaP, CWR22Rv1, MDA-PCa-2b, VCaP and LAPC-4 were purchased from American Type Culture Collection (Manassas, Va.). LNCaP/AR and PC3-PSMA were gifts from Charles Sawyers and Michel Sadelain, respectively (MSKCC, N.Y.). LNCaP, LNCaP/AR and CWR22Rv1 cells were maintained in RPMI1640 medium supplemented with 2 mM L-glutamine (Invitrogen, Carlsbad, Calif.), 1% penicillin-streptomycin (Invitrogen), and 10% heat-inactivated fetal calf serum (FCS) (Invitrogen). MDA-PCa-2b cells were grown in F12K medium containing 2 mM L-glutamine, 1% penicillin-streptomycin, 20% heat-inactivated FCS, 25 ng/mL cholera toxin (Sigma-Aldrich, St. Louis, Mo.), 10 ng/mL epidermal growth factor (BD Biosciences, San Jose, Calif.), 5 μM phosphoethanolamine (Sigma-Aldrich), 100 pg/mL hydrocortisone (Sigma-Aldrich), 45 nM selenious acid (Sigma-Aldrich) and 5 μg/mL insulin (Sigma-Aldrich). VCAP cells were maintained in DMEM medium supplemented with 2 mM L-glutamine, 1% penicillin-streptomycin and 10% non-heat-inactivated FCS. LAPC-4 cells were maintained in IMDM medium supplemented with 2 mM L-glutamine, 1% penicillin-streptomycin and 15% heat-inactivated FCS. All cell lines were kept at 37° C. in a 5% CO₂ atmosphere. 5α-dihydrotestosterone (DHT) was purchased from Wako Chemical USA (Richmond, Va.).

Numerous antibodies were used in these examples. Monoclonal antibody (mAb) anti-PSMA J591 was generated (Evans et al. (2011)). Additional antibody reagents included: mAb anti-AR (AR441), Rabbit anti-Human AR and goat polyclonal anti-GAPDH (Santa Cruz Biotechnology, Santa Cruz, Calif.), and mAb anti-PSA (Dako, Glostrup, Denmark). Mouse mAb anti-human beta-Actin was purchased from Thermo Scientific (Rockford, Ill.).

Fluorescence-activated cell sorting (FACS) analysis was also employed in these examples0. LNCaP, MDA-PCa-2b and LAPC-4 cells were seeded in 6-well plates (1×10⁵/well), grown overnight, and collected after trypsinization Immediately after 30 minute fixation with PBS containing 2% paraformaldehyde, the cells were incubated with murine anti-AR or anti-PSMA mAb in phosphate buffered saline (PBS) containing 1% bovine serum albumin (BSA) and 0.1% saponin (Sigma) for 1 hour, and then the cells were treated with fluorescein isothiocyanate (FITC)-conjugated sheep anti-mouse IgG (H+L, Jackson ImmunoResearch, West Grove, Pa.) antibody for 1 h. After washing with PBS containing 1% BSA+0.1% saponin, the cells were subjected to flow cytometric analysis (Becton Dickinson, Franklin Lakes, N.J.).

Immunohistochemistry studies were employed in these examples. Under an IACUC-approved protocol, CWR22Rv1 xenografts were established in BALB/c nude mice. At different time points post-castration, day 0 (non-castrate), weeks 1, 2, and 4, tumors were harvested, pre-cooled in liquid nitrogen, snap-frozen in OCT compound (Sakura Finetek U.S.A., inc., Torrance, Calif.) on dry ice, and stored at −800° C. Cryostat tissue sections were fixed in cold acetone (40° C.) for 10 minutes. The sections were washed in PBS. Peroxidase block (0.03%H₂O₂) was incubated for 5 minutes. After washing in PBS, humanized J591 (10 μg/ml in 1% bovine serum albumin) was incubated on the sections for 1 hour at room temperature. The diluent (1% BSA) was used as a negative control. Antibody binding was detected using rabbit anti-human Ig-peroxidase (Dako, Carpinteria, Calif.) followed by diaminobenzidine (Sigma-Aldrich Co., St. Louis, Mo.) as chromogen. The sections were counterstained with 10% hematoxylin.

### Overexpression or Silencing of AR

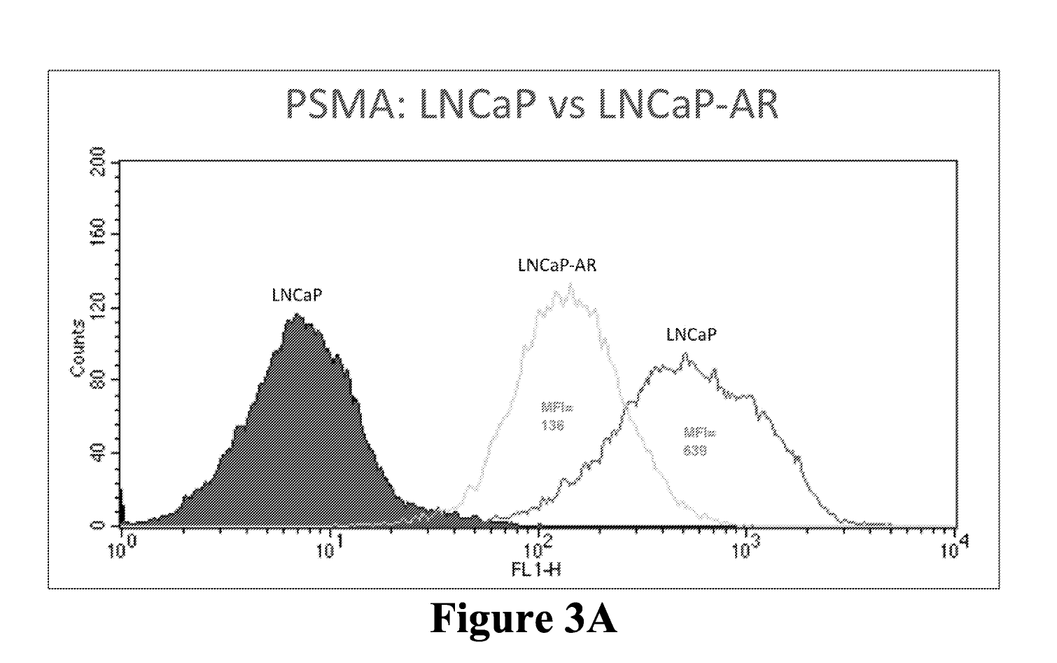

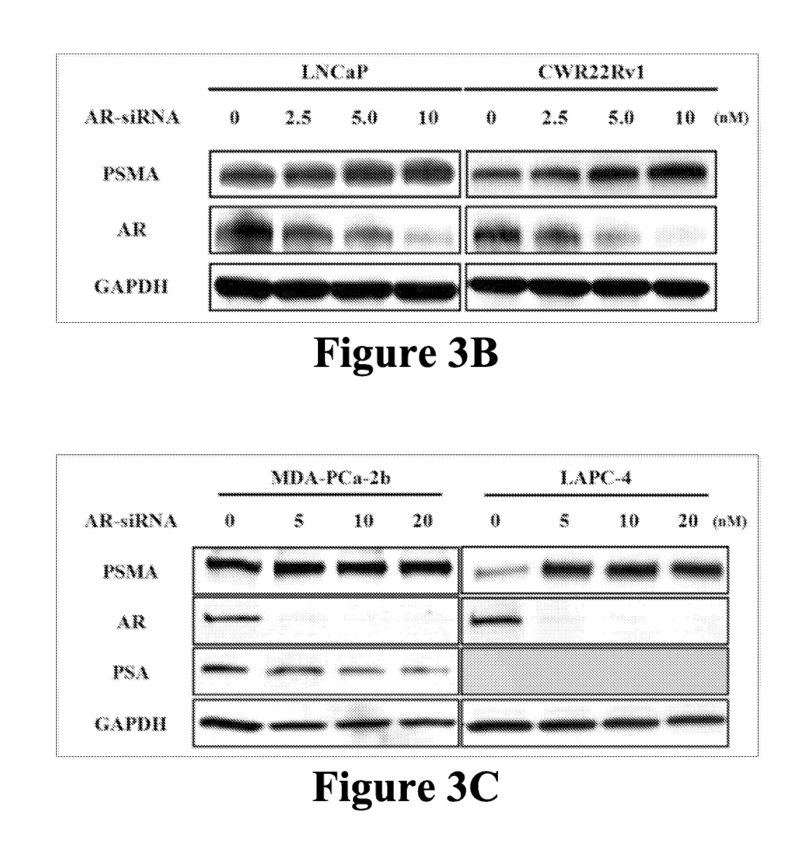

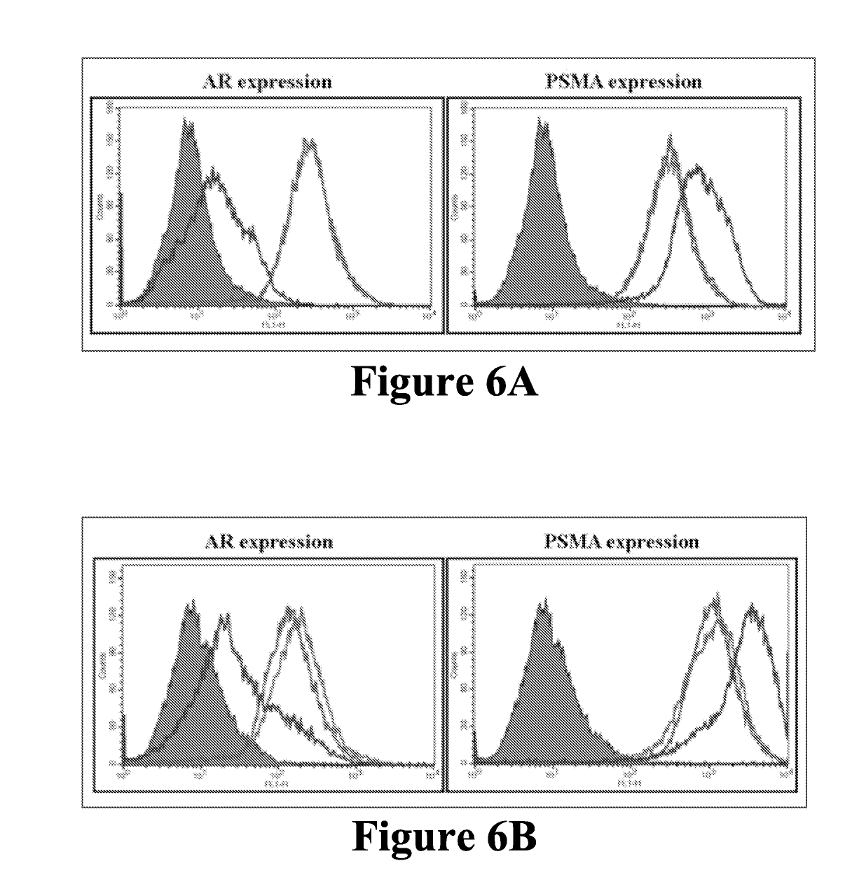

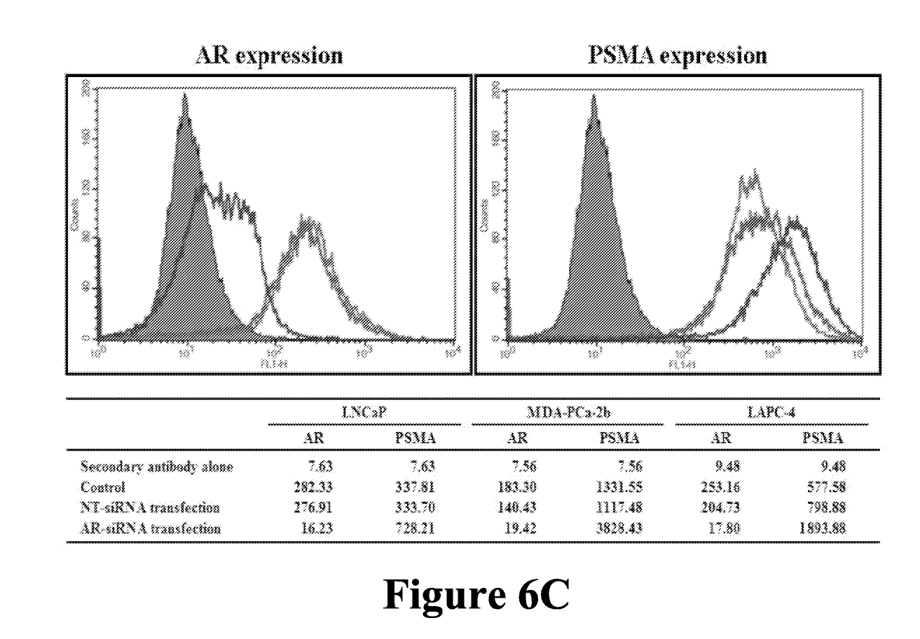

Transfection of the AR gene into LNCaP to over-express AR (i.e., LNCaP-AR) led to down-regulation of PSMA by approximately 80% (FIG. 3(a)). Conversely, silencing AR with siRNA led to a dose-dependent up-regulation of PSMA in all 4 cell lines tested (LNCaP, CWR22Rv1, MDA-Pca-2b and LAPC-4; (FIG. 3(b) and (c); FIG. 6). As expected, silencing AR led to a significant decrease in PSA secretion (data not shown).

RNA Interference was conducted as follows. Short interfering RNA (siRNA) duplexes specific to AR as well as non-targeting siRNA (NT-siRNA) were purchased from Dharmacon (Lafayette, Colo.). The AR-specific siRNA (AR-siRNA) sequence corresponds to the human AR site 5′-GACUCAGCUGCCCCAUCCA-3′. A NT-siRNA (5′-CCUACGCCACCAAUUUCGU-3′) was used as a control for the siRNA experiments. Following overnight incubation of the suspended cells transfected with varying doses of NT-siRNA or AR-siRNA using Lipofectamine RNAiMAX Reagent (Invitrogen) according to the manufacturer's instructions, media were changed with fresh media and the cells were incubated for the time indicated in Results and/or Figure Legends.

### Effect of Castration In Vivo

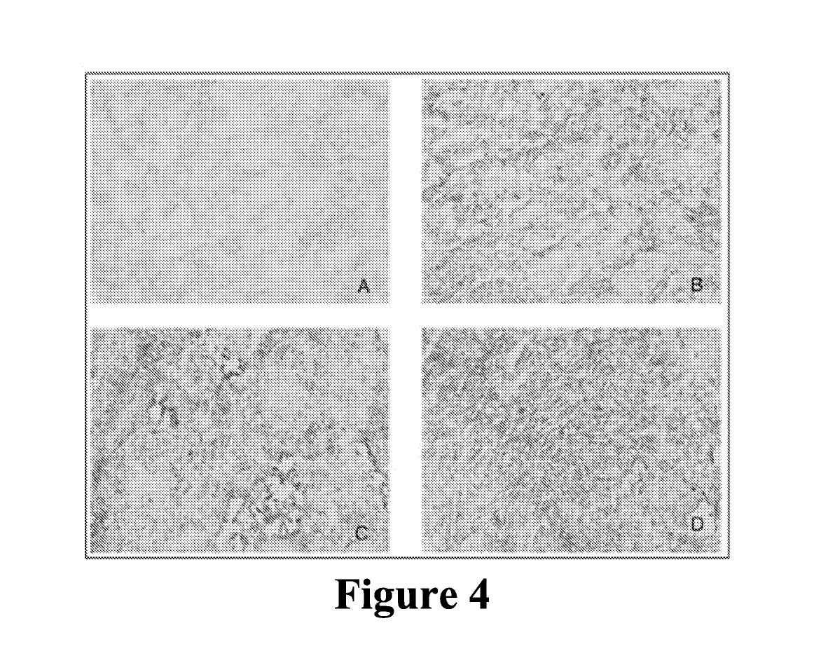

CWR22Rv1 xenografts growing in hormonally intact male nu/nu mice demonstrated low-level expression of PSMA (FIG. 4(a)) consistent with in vitro findings (FIG. 1, lane 5). Subsequent to surgical castration, the levels of PSMA expression rose progressively over the 4 week period of observation (FIGS. 4(b)-(d)).

### Anti-Tumor Activity of Castration Plus Anti-PSMA J591 Monoclonal Antibody-Drug Conjugate (ADC)

This study sought to determine the effect of the castration-induced up-regulation of PSMA on the anti-tumor response to a PSMA-targeted cytotoxic agent. CWR22Rv1 was chosen as it was established from an androgen-independent, castrate-resistant xenograft (Sramkoski et al. (1999); and Dagvadorj et al. (2008)) thereby allowing us to isolate the observed anti-tumor activity to the targeted agent plus any castration-induced PSMA up-regulation while eliminating a direct hormonal anti-tumor effect. In addition, as CWR22Rv1 grows rapidly, expresses relatively low levels of PSMA under physiological levels of androgen (FIGS. 1 and 4), expresses PSMA heterogeneously, and up-regulates PSMA only modestly relative to other PC cell lines (FIG. 1), it poses a near-worst case challenge to a PSMA-targeted agent.

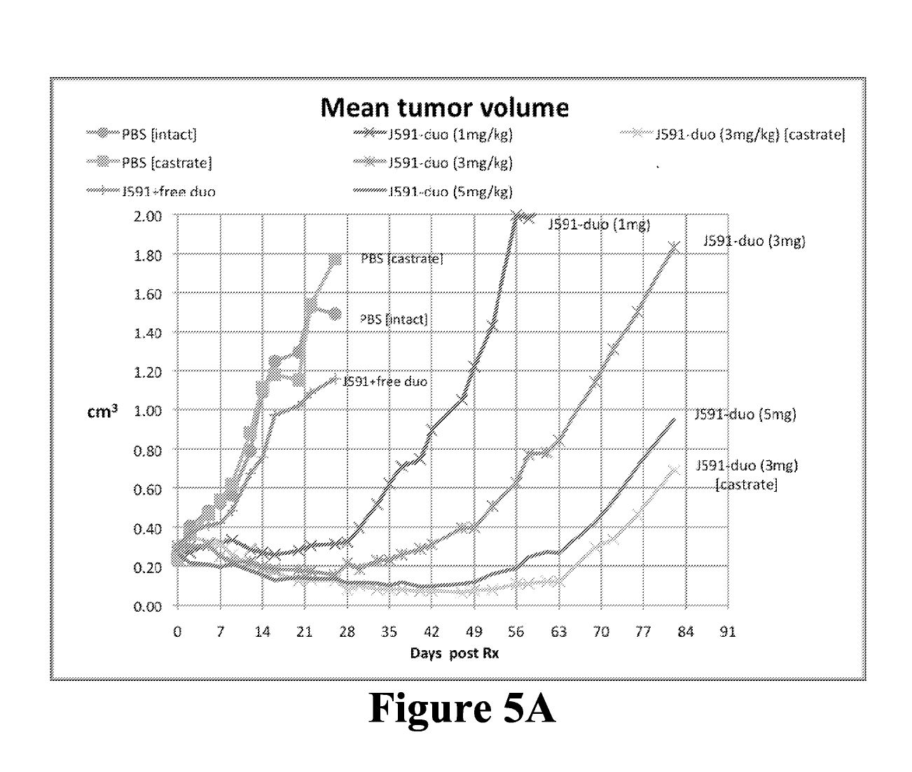

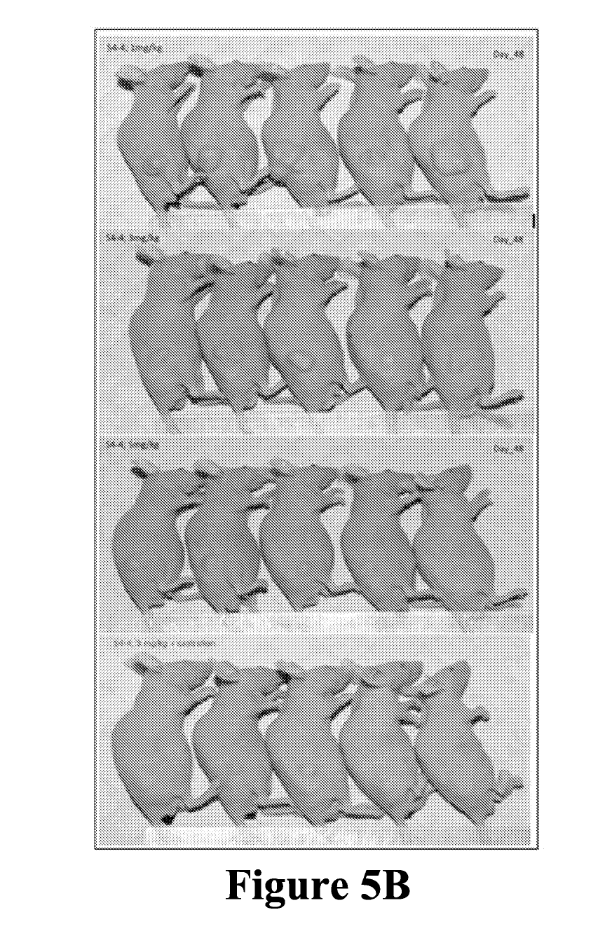

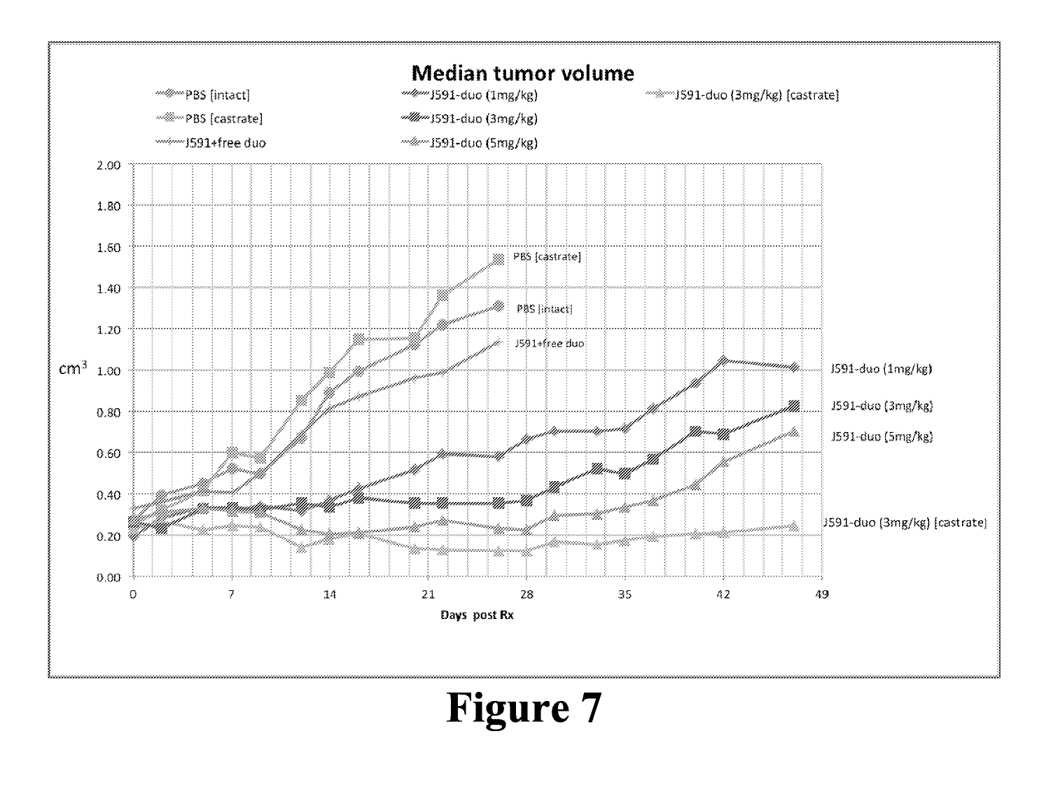

Under an IACUC-approved protocol, BALB/c nude mice were injected subcutaneously with 3×106 CWR22Rv1 cells suspended in matrigel (BD Biosciences, Bedford, Mass.). Two groups of animals were surgically castrated 9 days prior to injection of CWR22Rv1 cells. After establishment of growing tumors of approximately 250 mm³ (after about 5 days), animals were allocated into groups of 5 in such a manner that the mean tumor volume per group was approximately equal. Animals received an arbitrary 3 dose regimen at 2 week intervals (days 0, 14, 28) via tail vein injection. Controls consisted of PBS-treated intact mice, PBS-treated castrate mice, and mice treated with naked J591 anti-PSMA monoclonal antibody plus duocarmycin (unconjugated) at equivalent doses to the highest dose in the antibody-drug conjugate (ADC) groups. The PBS-treated group of intact mice demonstrated rapid tumor growth such that they required sacrifice by the end of the dosing period (day 28; FIG. 5). The castrate control group—castrated 14 days prior to onset of dosing—treated with PBS showed an identical growth curve to the intact mice, consistent with their castrate-resistant status, and also required sacrifice by day 28. The control group treated with unconjugated monoclonal antibody J591 and free duocarmycin showed minimal benefit from the treatment relative to the other control groups. Groups of animals received treatment with the J591/PSMA-targeted ADC at doses of 1, 3, or 5 mg/kg, as well as another group that was castrated and got 3 mg/kg. Tumor volume was calculated by the equation: 0.52×length×shortest width×shortest width. Tumor measurements were done in 2 dimensions thrice weekly with calipers. A clear dose-response effect is seen. While castration had no growth inhibitory effect on this castrate-resistant tumor model, the group treated with castration plus 3 mg/kg had an anti-tumor effect roughly equivalent to a 2-fold higher dose of ADC in the non-castrate animals. A second experiment using a different preparation of J591-duocarmycin showed an even greater than 2-fold enhancement in anti-tumor activity (FIG. 7). A third experiment using a different cytotoxin conjugated to J591 ADC showed a 2-fold enhancement in anti-tumor response (data not shown).

Taken together with the increase in PSMA expression seen post-castration (FIG. 4) and the increased tumor localization of the targeting antibody reported by PET (Evans et al. (2011)), this improved anti-tumor efficacy likely results from the higher PSMA expression driving greater targeting/internalization of targeted cytotoxin.

### Summary of Examples

These results show that androgen depletion led to an increase in PSMA expression in all 6 PSMA-positive PC cell lines tested. Similar PSMA up-regulation resulted from siRNA silencing AR suggesting that the effect was AR-mediated. Peak PSMA expression occurred in vitro at approximately 2 weeks post androgen-depletion. An inverse linear dose-response relationship was observed between androgen level and PSMA expression. Among different cell lines, castration-driven PSMA up-regulation ranged from 4-80-fold. Using CWR22Rv1 xenografts, significant up-regulation of PSMA was seen by immunohistochemistry over a 4 week period post-castration. Combining castration plus mAb J591 (anti-PSMA)-targeted ADCs led to synergistic anti-tumor responses even in castrate-resistant animal models. Thus, PSMA is a cell surface biomarker of androgen activity that can be readily identified and monitored by immunohistochemistry and/or in vivo imaging. Hormonal manipulation induces PSMA up-regulation even in castrate-resistant PC models and results in enhanced anti-tumor response. The inter-relationship of AR and PSMA make them a compelling target combination in PC.

### DESCRIPTION OF THE PREFERRED EMBODIMENTS

One aspect of the technology is use of an anti-prostate specific membrane antigen (PSMA) antibody or antigen binding fragment thereof for the preparation of a pharmaceutical composition for treating a prostatic cancer in a subject by administering to the subject an effective amount of said anti-PSMA antibody or antigen binding fragment thereof. In one aspect, the anti-PSMA antibody or antigen binding fragment thereof is conjugated to an anti-cancer agent. In another aspect, the anti-cancer agent is a cytotoxic agent. In another aspect, the subject is either castrate-resistant or is androgen-sensitive or androgen-responsive. In one aspect, the antibody or antigen binding fragment thereof is administered to the subject after measuring serum testosterone levels of 50 ng/ml or less. In another aspect, the antibody or antigen binding fragment thereof is administered to the subject within four weeks after initiating medical and/or surgical anti-androgen/castration therapy.

Another aspect of the technology is a method of treating a prostatic cancer, comprising administration of an anti-PSMA antibody or antigen binding fragment thereof conjugated to an anti-cancer agent to a subject. In a related aspect, the anti-cancer agent is a cytotoxic agent. In another aspect, the subject is castrate-resistant or is androgen-sensitive or androgen-responsive. In a related aspect, a first dose of the antibody or antigen binding fragment thereof is administered to the subject after measuring serum testosterone levels of 50 ng/ml or less. In another aspect, the first dose of the antibody or antigen binding fragment thereof is to be administered to the subject within four weeks after initiating medical and/or surgical anti-androgen/castration therapy.

In another aspect, the technology is directed to a method of treating prostate cancer comprising the steps of: (a) administering a medical and/or surgical anti-androgen/castration therapy to a subject having prostate cancer; and (b) administering to said subject an antibody or antigen binding fragment thereof that is capable of binding to the extracellular domain of PSMA. In another aspect, the antibody or antigen binding fragment thereof is conjugated to an anti-cancer agent. In another aspect, the anti-cancer agent is a cytotoxic agent. In yet another aspect, the cytotoxic agent is Lutetium-177. In a related aspect, the prostate cancer is castrate-resistant or is androgen-sensitive or androgen-responsive. In another aspect, the medical and/or surgical anti-androgen/castration therapy comprises hormonal therapy. In a related aspect, application of hormonal therapy enhances the effect of administration of the antibody or antigen binding fragment thereof that is capable of binding to the extracellular domain of PSMA. In another aspect, the hormonal therapy results in increased expression of PSMA by the prostate cells. In another aspect, the subject has been diagnosed with early stage non-metastatic cancer. In one aspect, the subject continues the hormonal therapy for at least 3-4 weeks. In another aspect, the medical and/or surgical anti-androgen/castration therapy comprises surgical castration.

In another aspect, the technology is directed to a method for identifying a test agent that increases the expression levels of PSMA on a prostate cancer comprising the steps of: (a) assessing the PSMA expression levels of a prostate cancer; (b) administering a dose of a test agent to said prostate cancer; (c) assessing the PSMA expression levels of said prostate cancer after administration with the test agent; and (d) comparing the PSMA expression levels of said prostate cancer before and after administration with the test agent. In a related aspect, the test agent is an agent that decreases androgen.

What is claimed is:

1. Use of an anti-prostate specific membrane antigen (PSMA) antibody or antigen binding fragment thereof for the preparation of a pharmaceutical composition for treating a prostatic cancer in a subject by administering to the subject an effective amount of said anti-PSMA antibody or antigen binding fragment thereof.

2. The use of claim 1, wherein the anti-PSMA antibody or antigen binding fragment thereof is conjugated to an anti-cancer agent.

3. The use of claim 2, wherein the anti-cancer agent is a cytotoxic agent.

4. The use of claim 3, wherein the subject is castrate-resistant.

5. The use of claim 3, wherein the subject is androgen-sensitive or androgen-responsive.

6. The use of any one of claims 1-5, wherein the antibody or antigen binding fragment thereof is administered to the subject after measuring serum testosterone levels of 50 ng/ml or less.

7. The use of any one of claims 1-5, wherein the antibody or antigen binding fragment thereof is administered to the subject within four weeks after initiating medical and/or surgical anti-androgen/castration therapy.

8. A method of treating a prostatic cancer, comprising administration of an anti-PSMA antibody or antigen binding fragment thereof conjugated to an anti-cancer agent to a subject.

9. The method of claim 8, wherein the anti-cancer agent is a cytotoxic agent.

10. The method of claim 9, wherein the subject is castrate-resistant.

11. The method of claim 9, wherein the subject is androgen-sensitive or androgen-responsive.

12. The method of any one of claims 8-11, wherein a first dose of the antibody or antigen binding fragment thereof is administered to the subject after measuring serum testosterone levels of 50 ng/ml or less.

13. The method of any one of claims 8-11, wherein a first dose of the antibody or antigen binding fragment thereof is to be administered to the subject within four weeks after initiating medical and/or surgical anti-androgen/castration therapy.

14. A method of treating prostate cancer comprising the steps of:
(a) administering a medical and/or surgical anti-androgen/castration therapy to a subject having prostate cancer; and
(b) administering to said subject an antibody or antigen binding fragment thereof that is capable of binding to the extracellular domain of PSMA.

15. The method of claim 14, wherein the antibody or antigen binding fragment thereof is conjugated to an anti-cancer agent.

16. The method of claim 15, wherein the anti-cancer agent is a cytotoxic agent.

17. The method of claim 16, wherein the cytotoxic agent is Lutetium-177.

18. The method of claim 17, wherein the prostate cancer is castrate-resistant.

19. The method of claim 17, wherein the prostate cancer is androgen-sensitive or androgen-responsive.

20. The method of any one of claims 14-19, wherein the medical and/or surgical anti-androgen/castration therapy comprises hormonal therapy.

21. The method of claim 20, wherein application of hormonal therapy enhances the effect of administration of the antibody or antigen binding fragment thereof that is capable of binding to the extracellular domain of PSMA.

22. The method of claim 21, wherein the hormonal therapy results in increased expression of PSMA by the prostate cells.

23. The method of claim 22, wherein the subject has been diagnosed with early stage non-metastatic cancer.

24. The method of claim 23, wherein the subject continues the hormonal therapy for at least 3-4 weeks.

25. The method of any one of claims 14-19 wherein the medical and/or surgical anti-androgen/castration therapy comprises surgical castration.

26. A method for identifying a test agent that increases the expression levels of PSMA on a prostate cancer comprising the steps of:
(a) assessing the PSMA expression levels of a prostate cancer;
(b) administering a dose of a test agent to said prostate cancer;
(c) assessing the PSMA expression levels of said prostate cancer after administration with the test agent; and
(d) comparing the PSMA expression levels of said prostate cancer before and after administration with the test agent.

27. The method of claim 26, wherein the test agent is an agent that decreases androgen.
